(19) United States
(12) Patent Application Publication
(10) Pub. No.: US 2025/0332293 A1
(43) Pub. Date: Oct. 30, 2025

(54) METHOD FOR MANUFACTURING BIOCOMPATIBLE NANOPARTICLES WITH A PEPTIDE CORE EXHIBITING SECOND- AND THIRD-HARMONIC SIGNAL GENERATION

(71) Applicants: Periklis PANTAZIS, Oberwil (CH); Ali Yasin SONAY, Zürich (CH); MAX-PLANCK-GESELLSCHAFT ZUR FÖRDERUNG DER WISSENSCHAFTEN E. V., München (DE)

(72) Inventors: Periklis PANTAZIS, Oberwil (CH); Ali Yasin SONAY, Zurich (CH); Daniel CRESPY, Rayong (TH); Katharina LANDFESTER, Mainz (DE)

(73) Assignees: Periklis PANTAZIS, Oberwil (CH); Ali Yasin SONAY, Zürich (CH); MAX-PLANCK-GESELLSCHAFT ZUR FÖRDERUNG DER WISSENSCHAFTEN E. V., München (DE)

(21) Appl. No.: 19/260,520

(22) Filed: Jul. 6, 2025

Related U.S. Application Data
(63) Continuation-in-part of application No. 16/337,570, filed on Mar. 28, 2019, now abandoned.

(30) Foreign Application Priority Data
Sep. 29, 2016 (EP) 16191538.4

(51) Int. Cl. A61K 49/00; G01N 21/64

(52) U.S. Cl. CPC A61K 49/0093; G01N 21/6428; G01N 2021/6439

(57) ABSTRACT

The invention relates to a method for preparing an aqueous suspension of biodegradable, water-suspendable nanoparticles (1) with a peptide core and a polymer shell, wherein the nanoparticles (1) each provide a second-harmonic signal and a third-harmonic signal upon illumination, the method comprising the formation a of a miniemulsion to allow for self-assembly of the peptide core and subsequent removal of the organic phase to allow formation of the polymer shell in the aqueous phase

### FIELD

The invention relates to a second-harmonic generating, water-suspensable nanoparticle, a method for preparing an aqueous solution of second-harmonic generating nanoparticles, a method for second-harmonic generation imaging and the use of such second-harmonic generating nanoparticles for imaging applications.

### BACKGROUND OF THE INVENTION

In optical imaging there are many techniques to generate and acquire an optical signal. One of these imaging techniques is the so-called second-harmonic generation imaging. Second-harmonic generation imaging is based on second-harmonic generation (SHG).

Another technique is the so-called third harmonic generation imaging, based on third harmonic generation (THG).

SHG as well as THG are nonlinear optical scattering-processes, in which due to a non-linear susceptibility term of the scattering material, two or three photons respectively with the same frequency result in a single, new photon with twice or three times the energy, and therefore twice or three times the frequency of the initial photons.

In general one may refer to SHG or THG as harmonic generation, HG. This effect is used in imaging applications like SHG or THG imaging. In these applications the HG, such as SHG or THG, probes respond with an HG signal under intense illumination. As the HG probe signal has a narrow signal profile and twice or three times the wavelength of the excitation light, the HG signal from the HG probe can be detected with minimal background.

Furthermore, due to the scattering nature of HG, HG imaging does not suffer bleaching or fluorescence intermittency of the probe, as the HG probe is not excited to higher energy levels (like in fluorescence imaging) from where bleaching and intermittency of the probe occurs.

SHG probes are known in the state-of-the-art, which are made of inorganic material that is not biodegradable, and therefore limiting the prospect of having them in clinical use or under clinical development (U.S. Pat. Nos. 8,945,471, 9,221,519).

Efforts have been made to prepare biocompatible SHG probes using peptides, however an inherent problem during preparation is that peptides self-assemble to SHG structures only in organic solvents [1]-[3] that aggregate in aqueous solutions, rendering them useless for most biological or medical applications.

With regard to THG probes little is known with regard to organic peptide probes. Therefore, the problem underlying the present invention is to provide a water-suspensable, biocompatible, biodegradable HG probe and a method for preparing an aqueous suspension comprising said HG probes.

This problem is solved by a method for manufacturing such an HG probe. Preferred embodiments are stated in the dependent claims.

### SUMMARY OF THE INVENTION

The invention provides for a biodegradable, biocompatible, water-suspensable nanoparticle, for generating a second- and/or third-harmonic light signal upon illumination with light; the nanoparticle comprising
a shell layer comprising or consisting of a biodegradable polymer, wherein the shell layer encloses
a plurality of peptides, wherein the plurality of peptides is structured such that a second-harmonic light signal and/or a third harmonic light signal is generated upon illumination of the nanoparticle with light.

### BRIEF DESCRIPTION OF THE FIGURES

FIG. 1 a schematic drawing of the preparation of a nanoparticle and a nanoparticle according to the invention;

FIG. 2 second-harmonic generation images of nanoparticles according to the invention;

FIG. 3 a schematic view of the self-assembling process of triphenylalanine;

FIGS. 4A-4D a time series of second-harmonic generation images of self-assembling triphenylalanine;

FIG. 5 a schematic drawing of a digestion of a self-assembled plurality of triphenylalanine by proteinase K;

FIGS. 6A-6D a time series of second-harmonic generation images of a digestion of self-assembled triphenylalanine;

FIGS. 7A-7D a time series of second-harmonic generation images of non-aggregating nanoparticles according to the invention; and

FIG. 8 molecular structures of cyclic oligopeptides that are suitable for assembling in a second-harmonic generating structure in the nanoparticle.

FIGS. 9A-9D SHG images for comparison of the methods and particles known from the state of the art with the nanoparticles according to the invention;

FIGS. 10A-10B SHG images showing the disassembly of oligopeptide assemblies known from the state of the art with regard to dilution;

FIGS. 11A-11B SHG images showing the disassembly of oligopeptide assemblies known from the state of the art with regard to the pH-value of the solvent;

FIGS. 12A-12B SHG images showing the stability of nanoparticles according to the invention with regard to dilution;

FIGS. 13A-13D SHG images showing the stability of nanoparticles according to the invention with regard to the pH-value of the solvent;

FIGS. 14A-14D shows X-ray diffraction analysis of the nanoparticles manufactured according to the invention;

FIGS. 15A-15B shows the nanoparticles generating a THG signal;

FIGS. 16A-16E shows the stability of the nanoparticle with regard to various pHs;

FIGS. 17A-17C shows absence of protein-level toxicity of the nanoparticles;

FIGS. 18A-18B shows absence of cell toxicity;

FIGS. 19A-19C shows a comparison to non-organic nanoparticles;

FIGS. 20A-20D shows emission of nanoparticles having different peptide cores;

FIGS. 21A-21B shows the polarization dependence of the second harmonic signal of the nanoparticle.

### DETAILED DESCRIPTION

The peptides may be selected from the group consisting monopeptides, i.e. mono-amino acids, or oligopeptides, i.e. peptides comprising more than one amino-acid.

According to another embodiment of the invention, the monopeptides may be selected form the group consisiting of Phe, Tyr, Trp.

Such a nanoparticle advantageously solves the problem according to the invention, as it is water-soluble and biocompatible. The water-suspensability of the nanoparticles is particularly achieved by the shell layer that comprises a biodegradable polymer, wherein the shell layer provides water-suspensability to the water-insoluble or not-well soluble, structured plurality of peptides.

Another advantage of the nanoparticle according to the invention is that the nanoparticle does not tend to aggregate with other nanoparticles of the same kind in an aqueous solution, wherein the pure peptides suspended in an aqueous solution might aggregate over time.

Self-assembling peptides can form large scale assemblies that can generate SHG signal and/or the THG signal, as the orientation of the assembly lacks inversion symmetry. The SHG signal intensity is proportional to the number of peptides employed for the assembly, which is related to the size of the assembly.

Consequently, smaller nanoparticles particularly generate a smaller SHG signal. This invention provides a biodegradable, biocompatible, water-suspensable nanoparticle, particularly in the diameter range of 50 nm to 200 nm, that surprisingly does generate a sufficiently high SHG signal for example for use in microscopy applications.

Large-scale assemblies, as for example disclosed in [4] (particle sizes ˜1 μm to 10 μm), typically need to satisfy phase-matching conditions and their signal propagates predominantly in the forward direction, which limits the imaging capabilities of large assemblies. Moreover, these large scale assemblies exhibit a particularly broad size range is due to the dynamic nature of the assembly process, which can assemble and disassemble in response to environmental changes.

Given the preference of peptides to form aggregates, the nanoparticle according to the invention solves this problem by providing a coating that ensures that the peptide assemblies do not aggregate and growth is restricted. This way, a precise control of the size range and distribution of the nanoparticles is achieved.

Furthermore, suitable peptides for the nanoparticle that generate an SHG signal upon illumination particularly comprise or consist of peptides that self-assemble into beta sheets and 7E-7E stackings through crystallization.

In contrast, the example peptide LIVAGK disclosed in [4] relies on decreasing amino acid sizes within the peptide sequences, which do not self-assemble or generate SHG signal after encapsulation.

Furthermore the peptides disclosed in [4] require a specific peptide concentration and pH value in order to self-assemble. Thus, the assembly process of peptides in [4] is vulnerable to environmental changes. Upon dilution or pH-change these assemblies disassemble rapidly. The instability of the peptide assemblies taught in [4] render them impractical for any tracking experiment within cells and tissues, where imaging probes typically encounter lower pH values for an extended period of time.

In contrast, the nanoparticles according to the invention remain stable under such environmental changes.

Given that the peptide assemblies in [4] are not coated with any polymer, any attempt to functionalize and target this assembly to a cell or protein of interest would necessitate adding functional groups on the peptides, which would disrupt the assembly process.

The shell layer of the nanoparticle allows the nanoparticle i) to be targeted to sites of interest with great precision and ii) to protect the assembled peptides from dissociation or disassembly. Hence, the nanoparticles according to the invention are particularly applicable as imaging agents in various biomedical applications.

[4] specifies a restricted sequence order in order to generate the SHG-generating peptides. Given that the SHG signal was measured in a hydrogel, such restriction is necessary. In contrast, the peptides suitable for the nanoparticles according to the invention, do not suffer from such restrictions and their self-assembly particularly depends on their crystal structure as opposed to their shape.

This difference particularly shows that the SHG generating mechanism in [4] is different to the SHG generation of the nanoparticles according to the invention as will be explained in the following.

The peptides disclosed in [4] require certain shapes and sizes as well as sequences to generate an SHG signal. This indicates that their crystallisation plays little role in SHG generation.

SHG signal generation of a nanoparticle according to the invention particularly relies on SHG generated by crystal unit cells.

The shell layer might not necessarily be understood as a complete and tight coating of the peptides, but it might comprise pores and openings, through which particularly an organic solvent can evaporate. The shell layer or parts of the shell layer particularly extend to the inner part of the nanoparticle, wherein other parts of the layer might extend to the environment of the nanoparticle, e.g. into an aqueous solution. The exact structure of the shell layer is of no great importance, as long as it renders the nanoparticle water-suspensable, biodegradable and non-aggregating.

It is the structured plurality of peptides in the nanoparticle that gives rise to the SHG signal or the THG signal upon illumination with light. The capability of the structured plurality of peptides of generating a second-harmonic signal in turn roots in the lack of the inversion symmetry of the structured plurality of peptides. Thus, without a structured plurality of peptides or with a centrosymmetric structure of peptides no appreciable SHG signal can be generated.

Similarly, the capability to generate a THG signal upon illumination lies in the choice of peptide and their specific form of the self-assembled structured plurality of peptides that in order to generate also the SHG signal lacks the inversion symmetry.

A nanoparticle according to the invention has a diameter ranging particularly between 10 nm and 5 μm, particularly between 50 nm and 1 μm, more particularly between 100 nm and 200 nm.

### “Biocompatibility”

A biocompatible nanoparticle in the context of the present invention is particularly non-toxic to biological tissue, cells or a living body, as long as it does not comprise additional and specific epitopes or substances that are designed for triggering for example cell death or for altering cellular signalling pathways.

### “Biodegradable”

A biodegradable nanoparticle according to the invention refers the property of the nanoparticle of being degradable particularly by specific enzymes, bacteria, fungi or cells.

Illustrative biodegradable materials suitable for use in the practice of the invention include naturally derived polymers, such as acacia, gelatin, dextrans, albumins, alginates/starch, and the like; or synthetic polymers, whether hydrophilic or hydrophobic.

As used herein, the terms “biodegradable” and “biocompatible” therefore denote any synthetic or naturally-derived material that is known as being suitable for uses in the body of a living being, i.e., is particularly biologically inert and physiologically acceptable, non-toxic, and, in the context of the present invention, is biodegradable in the environment of use, i.e., can be resorbed by the body or degraded by specific enzymes or bacteria.

### “Oligopeptide”

An oligopeptide consists of at least two amino acids and comprises particularly less than 100 amino acids that are chemically linked. Particularly oligopeptides consisting of two, three, four, five, six or seven amino acids are suitable for the invention, as long as they are capable of forming a structured plurality that generates an SHG signal or the THG when comprised by the nanoparticle and when illuminated with light.

It is noted that not all structured pluralities of peptides, particularly oligopeptides are capable of generating a second-harmonic signal or THG signal, even though theoretically they should do so. Thus, each different monopeptides and oligopeptide has to be tested for the SHG and THG property separately. Furthermore, it is noted that even if the peptides assemble in a second-harmonic generating structure, it is observed that after preparation of the nanoparticle according to the invention, said nanoparticle might not generate the SHG signal anymore. Also here, a thorough testing of various oligopeptides is necessary.

### “Structured”

The term “structured” refers to the fact, that the plurality of peptides is arranged at least area by area in an assembly that exhibits a certain regularity or repeated pattern and that the structured plurality of peptides is particularly not arranged in a random coil configuration. Nonetheless it is possible that a fraction of enclosed peptides in the nanoparticle is not adopting a structured configuration and does not produce an SHG signal upon illumination. This fraction is then simply not considered to be part of the structured plurality of peptides. The same may hold true for THG generation.

Further, structured peptides are particularly those peptides that crystallize in non-centrosymmetric cells, e.g. cells exhibiting C₂-symmetry. This may be confirmed by way of polarization-resolved SHG fitting.

Furthermore, the structured peptides are particularly not arranged in a lattice formed exclusively by chemical bonds, such as covalent bonds.

As stated above, the structured plurality of peptides is particularly lacking inversion symmetry, such that the structured plurality of peptides is capable of second-harmonic generation and/or third harmonic generation.

The structured plurality of peptides generates a second-harmonic signal upon illumination with light, wherein the excitation light comprises preferably wavelengths in the range of 400 nm to 2000 nm. Further, the structured plurality of peptides may generate a third-harmonic signal upon illumination with light, wherein the excitation light comprises preferably wavelengths in the range of 400 nm to 3000 nm.

The SHG strength or SHG susceptibility of the nanoparticle particularly depends on the number and assembly orientation of the structured peptides comprised by the nanoparticle. The structured plurality of peptides particularly comprises more than 100 peptides.

According to another embodiment of the invention, the plurality of peptides comprises or consists of self-assembling peptides, wherein the plurality of structured peptides is arranged in a self-assembled structure.

This embodiment is particularly advantageous as the preparation of nanoparticles is greatly simplified by using self-assembling peptides.

### “Self-Assembling”

The term “self-assembling” in the context of the invention refers to the property of the peptides that the peptides assemble in predefined structures or assemblies, wherein structure of the self-assembly particularly depends on the specific amino acid sequence of the peptides and/or the specific mixture or mixing proportion of different peptides as well as the solvent properties. The self-assembling process of the peptides is particularly triggered by the peptide concentration.

An example of a self-assembling peptide is the tripeptide Phe-Phe-Phe. Said tripeptide, triphenylalanine, is capable of self-assembling into nanorods.

According to another embodiment of the invention, the plurality of peptides is crystalized in crystal unit cells, wherein the SHG signal is generated from the crystal unit cells upon illumination of the nanoparticle.

The expression “encapsulating and maintaining the structured plurality of peptides” particularly refers to the polymer shell enclosing the peptide core in a manner that prevents disassociation of the peptides core in the aqueous phase while further preventing the peptide core to loose its second and third harmonic generating structure.

According to another embodiment of the invention the plurality of peptides comprises oligopeptides that are selected form the group consisting of:
cyclo(-D-Trp-Tyr), wherein “D-Trp” refers to the D-amino-acid form (D enantionmer) of Trp,
Trp-Phe,
Phe-Phe-Phe,
Phe-Phe,
Ala-Ala-Ala-Ala-Ala (SEQ ID NO 01),
cyclo(-Phe-Phe),
Leu-Phe, and/or
Leu-Leu,
   wherein three-letter codes are used to refer to the specific amino acid sequence of the respective oligopeptide. The prefix “cyclo” indicates that the oligopeptide exhibits a cyclic structure. These oligopeptides are capable to self-assemble in a structure that is capable of second-harmonic generation and maintaining its second-harmonic generating property also when enclosed by the shell layer and when the nanoparticle is suspended in an aqueous solution.

According to another embodiment of the invention the plurality of peptides comprises at least one structured and shape-persistent region exhibiting a high degree of internal order. Said shape-persistent region is capable of second-harmonic generation and may be capable to also generate the third harmonic signal.

The degree of internal order of the structured plurality has to be so high that an SHG signal is generated upon illumination of the nanoparticle. Thus, ideally all enclosed peptides are part of a particularly self-assembled structure, wherein also assemblies are included in the meaning of the invention, where the plurality of structured peptides is assembled in a plurality of structures, wherein at least one of these structures generates a second-harmonic signal upon illumination. Thus, particularly also nanoparticles comprising a plurality of structured pluralities of peptides, e.g. a plurality of nanorods or any other second-harmonic generating structure are to be summarized under the claimed invention.

These particularly identical structures might have different orientations within the nanoparticle, which is particularly advantageous with regard to its SHG susceptibility.

A high degree of internal order refers particularly to the fact that the majority of peptides are part of a structured plurality of peptides and particularly only less than half of the enclosed peptides are in an un-ordered state, such as a random coil configuration.

A high degree of internal order also refers to the fact that particularly more than 50% of the structured plurality of peptides are ordered in a non-centrosymmetrical structure. All centrosymmetrically structured pluralities, such as for example a random coil configuration, lead to a cancellation of the SHG signal due to destructive interference.

### “Shape-Persistent”

The term “shape-persistent” refers to the property of the nanoparticles, that the particularly self-assembled structures do not spontaneously change their shape or organization, but generally maintain their shape until the nanoparticle is degraded.

According to another embodiment of the invention the plurality of structured peptides is structured by means of non-covalent interactions, particularly by hydrophobic interactions and/or hydrogen bonds. Structures formed by non-covalent interactions are particularly useful for preparing the nanoparticle according to the invention, as no further chemical reactants are needed in order to obtain such particularly self-assembling structures. Furthermore these structures tend be biodegradable.

According to another embodiment of the invention, the biodegradable polymer is one of:
a poly(L-lactide) (PLLA) (CAS Nr:26100-51-6),
a polyglycolide and a polylactic polyglycolic copolymer
a poly(ε-caprolactone)-polyether (CAS-Nr: 24980-41-4),
a polycaprolactone,
a poly[(D,L-lactide)-co-glycolide](PLGA),
a polyacrylamide,
a poly(orthoester),
a biodegradable polyurethane,
a polycyanoacrylate polymer,
a poly(γ-glutamic acid) (γ-PGA) (CAS-Nr: 25736-27-0),
a phenylalanine ethyl ester.

These polymers are particularly suitable for forming a biodegradable and biocompatible shell layer of the nanoparticle.

According to another embodiment of the invention, the nanoparticle is digestable by an enzyme, a proteinase, particularly by proteinase K (CAS-Nr: 39450-01-6), particularly after removing any surfactant. Therefore, the nanoparticles according to the invention are particularly biodigestable. This digestion might occur at a slower rate than a digestion of peptides alone, i.e. without a polymer shell.

The property of being biodigestable is particularly important and advantageous when the nanoparticle is to be used in biological imaging or medical applications.

According to another embodiment of the invention the nanoparticle particularly the polymer comprises a fluorescent compound, wherein said fluorescent compound is particularly a covalently linked fluorescent dye. A nanoparticle comprising a fluorescent compound enables the co-localization of the fluorescence signal and the SHG signal stemming from the same nanoparticle and thus allows the monitoring of the preparation process of the nanoparticle.

The problem according to the invention is also solved by a method for preparing an aqueous suspension of biodegradable, water-suspended nanoparticles, wherein the nanoparticles generate a second-harmonic signal and a third harmonic signal upon illumination with light, comprising the steps:
providing an organic phase comprising an organic, particularly water-immiscible, solvent with peptides and biodegradable polymers and/or monomers, wherein the monomers form the polymer when polymerized, wherein the peptides in each nanoparticle are assembled, particularly crystallized in at least one structure once the nanoparticle is prepared, wherein said structure generates a second-harmonic signal and/or a third harmonic signal when illuminated with matching excitation light,
providing a continuous aqueous phase comprising an aqueous solution with a surfactant, particularly a hydrophilic surfactant,
preparing a miniemulsion of the organic phase and the aqueous phase by mixing the organic phase and the aqueous phase, wherein the miniemulsion comprises droplets, particularly with a diameter between 10 nm and 5 μm, more particularly with a diameter between 20 nm and 500 nm, of the organic phase emulsified in the aqueous phase,
removing the organic solvent from the miniemulsion such that the nanoparticles are formed.

According to the definition of the UIPAC, a miniemulsion is an emulsion in which the droplets of the dispersed phase have diameters in the range from approximately 50 nm to 1 μm. In the case of the present invention, the organic phase is the dispersed phase, the continuous phase corresponds to the aqueous phase and also an emulsion with droplet sizes up to 5 μm is referred to as a miniemulsion.

In particular, the miniemulsion is formed as nanoemulsion, particularly having droplet sizes in the range between 30 nm and 300 nm.

The peptides self-assemble in the structured plurality of peptides during preparation of the nanoparticle. The self-assembling may start as early as the droplets have formed in the miniemulsion, as the local concentration of the peptides in the droplets is then high enough to trigger the assembling process. The self-assembling is particularly also happening during evaporation of the organic solvent from the miniemulsion, as the concentration of peptides in the shrinking droplets during evaporation increases even more.

The self-assembling may comprise the process of crystallizing of the peptides into the at least one structured plurality of peptides to form the peptide core.

According to another embodiment of the invention, the structured plurality of peptides self-assembles and crystallizes in situ within the droplets.

According to another embodiment of the invention the organic phase contains mixed stabilizers, for example an ionic surfactant, such as sodium dodecyl sulfate (n-dodecyl sulfate sodium) and a short aliphatic chain alcohol (referred to as co-surfactant) for colloidal stability, or a water-insoluble compound, such as a hydrocarbon (referred to as a co-stabilizer) limiting diffusion degradation. By adding a mixed surfactant, a co-surfactant and/or co-stabilizer the mini-emulsion according to the invention can be stabilized for several days.

The miniemulsion can be prepared by applying shear-forces to the mixed organic and aqueous phase.

According to another embodiment of the invention the miniemulsion is prepared by applying shear-forces to the organic phase and to the aqueous phase, particularly after mixing the two phases coarsely, particularly by shaking, wherein the shear-forces are applied by sonication.

According to another embodiment of the invention, by removing the organic solvent from the miniemulsion the polymer precipitates to form the shell of the nanoparticle.

According to another embodiment of the invention, the organic solvent is removed by evaporating the organic solvent from the miniemulsion, wherein evaporation is particularly achieved by heating and/or by reducing the surrounding atmospheric pressure.

The organic solvent can be removed to great extent from the miniemulsion by evaporation, such the miniemulsion transforms in an aqueous suspension comprising the aqueous, continuous phase and the water-suspended, biodegradable nanoparticles.

Thus, the miniemuslion is not an emulsion anymore after the organic solvent is removed, but it is converted in an aqueous suspension of nanoparticles.

According to another embodiment of the invention, the organic solvent is chloroform, the peptide is or comprises the tripeptide Phe-Phe-Phe and the polymers comprise or consist of Poly-Lactic Acid or the monomers comprise or consist of lactat and the aqueous solution comprises sodium dodecyl sulfate (n-dodecyl sulfate sodium) as surfactant.

Nanoparticles made from this composition are robust and reliable in preparation.

According to another embodiment of the invention the monomers are polymerized in a polymerization step.

This step might be carried out simultaneously or subsequently to the removal of the organic solvent. The polymerization step is providing increased stability for nanoparticles that otherwise would consist only of monomers.

The problem according to the invention is further solved by a method for second-harmonic generation and/or third harmonic imaging of a sample comprising a nanoparticle according to the invention, comprising the steps of:
providing a sample comprising at least one nanoparticle according to the invention, wherein the sample is particularly a biological sample such as cells, a tissue preparation, tissue or a living organism,
illuminating the sample with light comprising a first wavelength, particularly 1064 nm or 1596 nm,
detecting light at half the wavelength of the first wavelength, and particularly filtering the illumination light.

The sample is particularly obtained from a person or an animal. The at least one nanoparticle is particularly provided to the sample after the sample is obtained from the person or animal.

The problem according to the invention is further solved by a use of the nanoparticle according to the invention, in second-harmonic generation imaging of biological samples, cells, tissue preparations, tissue or living organisms, particularly applying the method for second-harmonic imaging.

Also here, the biological samples, cells tissue preparations, tissue or living organisms are particularly obtained from a person or an animal prior to the provision of nanoparticle to the sample and particularly prior to imaging.

Particularly, exemplary embodiments are described below in conjunction with the Figures. The Figures are appended to the claims and are accompanied by text explaining individual features of the shown embodiments and aspects of the present invention. Each individual feature shown in the Figures and/or mentioned in said text of the Figures may be incorporated (also in an isolated fashion) into a claim relating to the device according to the present invention.

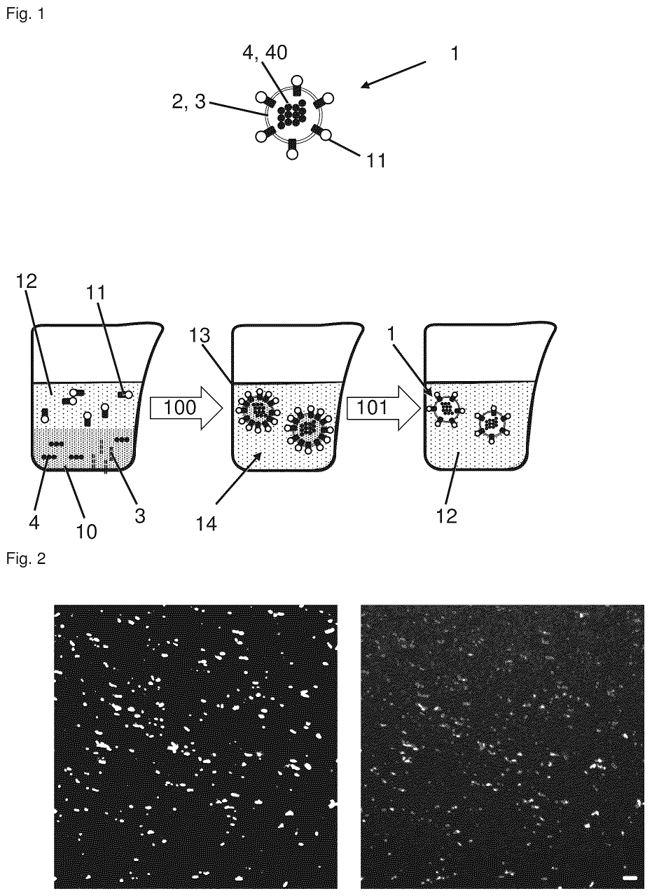

FIG. 1 schematically shows the preparation steps for obtaining a nanoparticle 1 according to the invention.

In step A) a continuous phase, here the aqueous phase 12 comprising water and a surfactant 11 is provided. The continuous phase 12 rests on top of a dispersed phase, here the organic phase 10, which comprises chloroform, a preformed polymer 3, in this case PLLA, and peptides 4, in this case triphenylalanines 41, capable of self-assembling into a second-harmonic generating structure, particularly to a nanotube or a nanorod, that generates a second-harmonic signal upon illumination with light. In this example the polymer 3 is linked to a fluorescent dye, in the present case to Alexa 488, such that co-localization of the fluorescence signal and the second-harmonic signal becomes possible. This is particularly advantageous to monitor a successful preparation of second-harmonic generating nanoparticles 1—a co-localized signal indicates that there are nanoparticles 1 suspended in the solution comprising polymer 3 and peptides 4 assembled in a second-harmonic generating conformation.

In step B) the continuous phase 12 and the dispersed phase 10 are mixed together and sonicated 100 for several minutes such that droplets 13 comprising a plurality 40 of peptides 4, the polymer 3 and the organic solvent are formed. Such emulsion is referred to as a miniemulsion 14.

The peptides 4 are located on the inside of the droplets 13, where the organic solvent is encapsulated by the polymer 3. The polymer 3 thus forms a shell 2 for the peptides 4. The droplets 13 are dispersed in the continuous phase 12.

The miniemulsion 14 obtained by the sonication 100 step B) is then heated (step C), such that the organic solvent evaporates 101 from the droplets 13. Once evaporated, the droplets 13 are gone and the solid nanoparticles 1 suspended in the continuous phase 12 remain. The nanoparticles 1 comprise the polymer shell 2 with a fluorescent dye, wherein the polymer shell 2 encapsulates the self-assembled peptides 4. Upon illumination the nanoparticle 1 will generate a second-harmonic and third harmonic signal from the illumination light due to the self-assembled structured plurality 40 of peptides 4.

One important property of these nanoparticles 1 is that besides being capable of generating a second-harmonic signal and the third harmonic signal, said nanoparticles 1 also have the property that they do not aggregate over time. This is particularly useful for targeting and imaging applications that require inert probes for aqueous solutions.

Further, the nanoparticles are non-toxic for cells and organisms.

As the polymer shell 2 is biodegradable the nanoparticles 1 themselves are biodegradable.

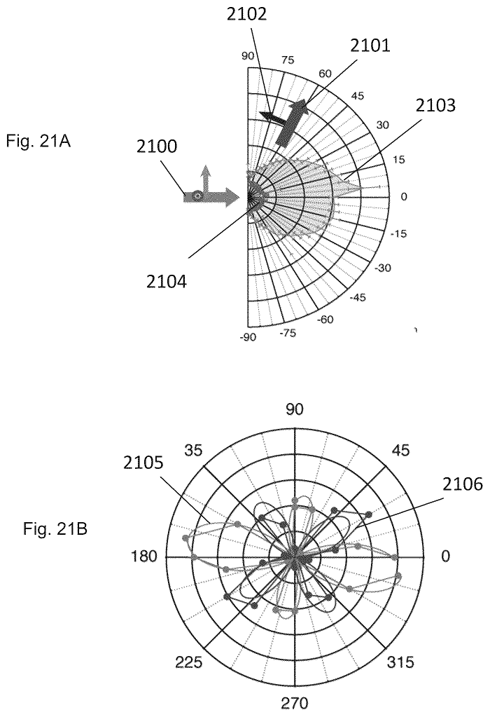

FIG. 2 shows images of second-harmonic generating nanoparticles 1 that comprise a fluorescently labelled polymer 3 according to the invention. On the left panel the SHG image is depicted, wherein on the right panel of FIG. 2 the fluorescence image is shown. The high degree of co-localization of the SHG signal and the fluorescence signal can be readily seen. The high degree of co-localization in turn indicates a successful formation of second-harmonic generating nanoparticles 1 comprising a polymer shell 2. To confirm the second-harmonic generation, polarization-resolved SHG measurements under PPP and PSS configurations have been performed. The angular dependence of the SHG intensity fits a C₂ non-centrosymmetric symmetry, confirming that the peptide core crystallises in an ordered, second-harmonic generating structure or lattice. These polarization resolved measurements are depicted in FIG. 21.

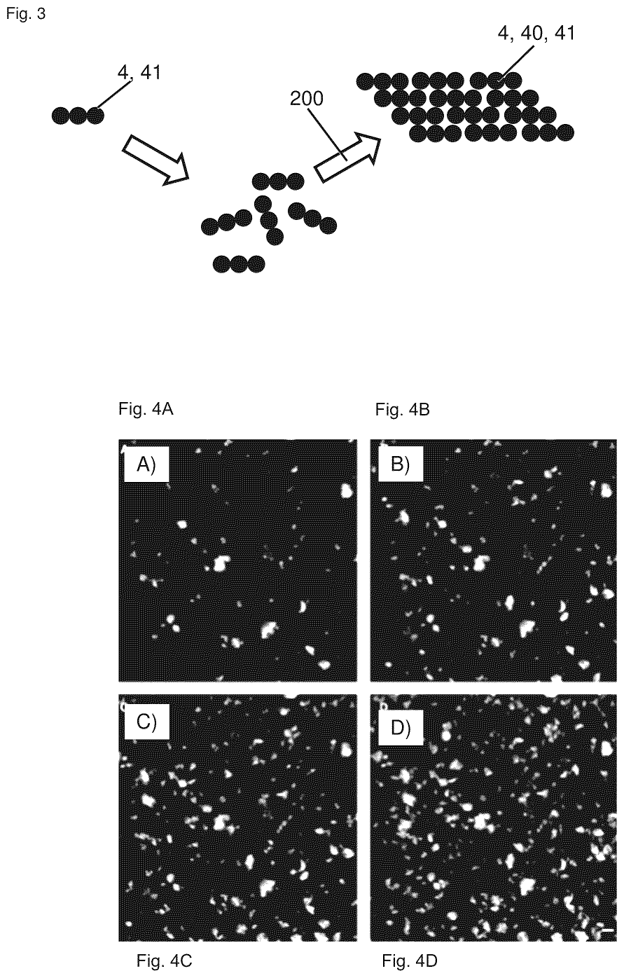

FIG. 3 schematically shows how a plurality 40 of peptides 4, in this case triphenylalanines 41 (Phe-Phe-Phe), self-assemble 200 to a structure, namely a nanorod, that due to its lack of inversion symmetry is capable of second-harmonic generation.

FIG. 4 shows a time series of SHG images of triphenylalanine 41 peptides 4 dissolved deionized water containing 1% Pluronic surfactants. The time series shows the effect of aggregation over time of uncoated nanoparticles, i.e. nanoparticles that do not comprise a polymer shell. Image A) has been taken at time zero minutes, image B) has been taken after 10 minutes, image C) has been taken after 20 minutes and image D) has been taken 40 minutes after dissolving the peptides.

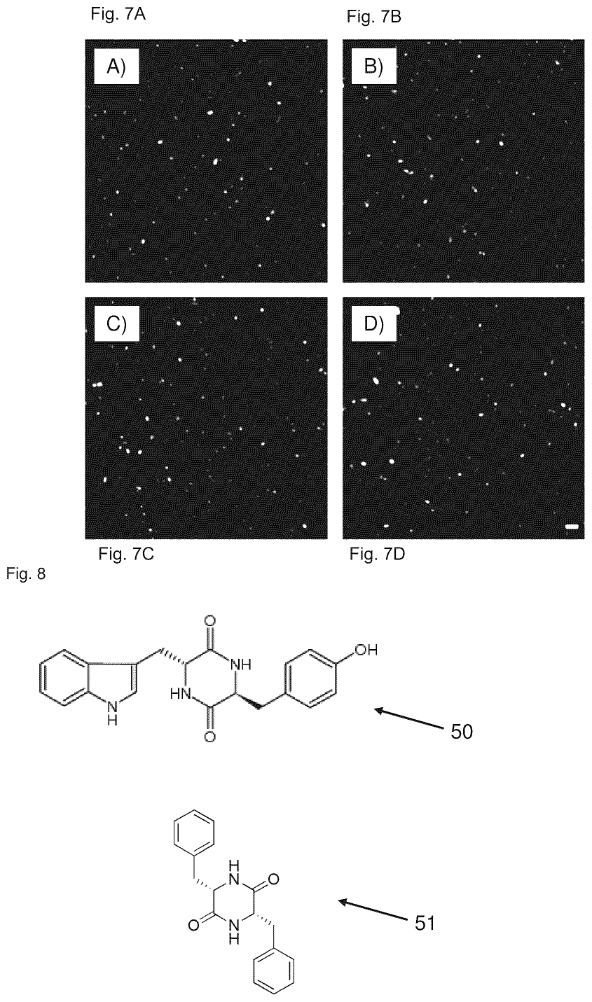

As can be seen, the overall SHG signal increases, which indicates the formation of more and larger self-assembled nanostructures that are capable of second-harmonic generation. It also indicates that the cluster size is increasing with time, which points to a progressing aggregation of self-assembled structures. Therefore, the polymer shell is particularly advantageous in order to inhibit a progressing aggregation that would take place otherwise. The aggregation-inhibiting effect of the polymer shell 2 is shown in FIG. 7.

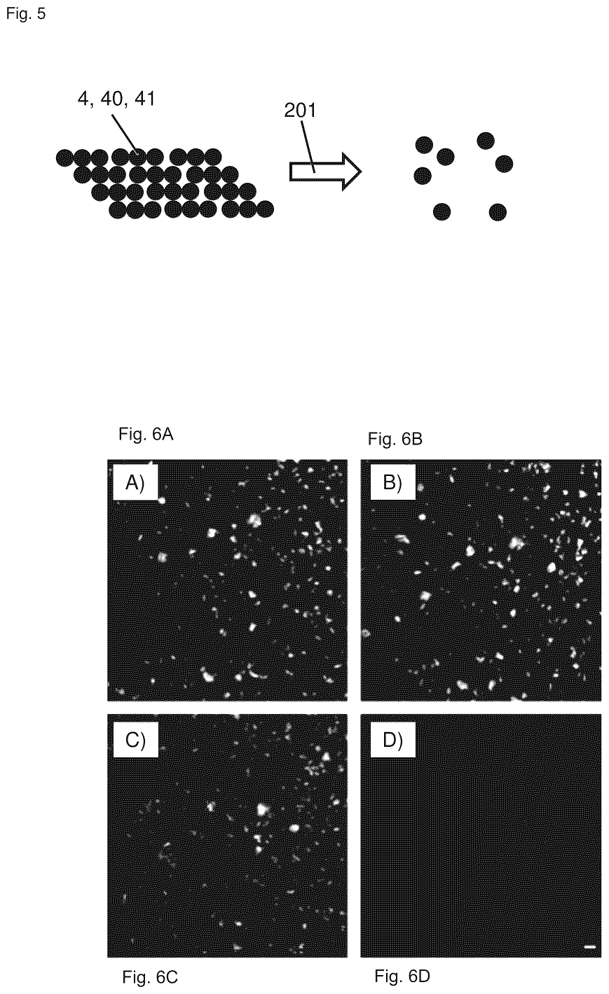

FIG. 5 shows a schematic drawing of a digestion 201 of a self-assembled structure of peptides 4 comprising triphenylalanine 41, wherein the structures are not enclosed by a polymer shell 2. The digestion 201 is facilitated by a proteinase K.

The proteinase K digests the self-assembled structure into single phenylalanines.

In FIG. 6 the corresponding SHG images of a digestion 201 of self-assembled structures of triphenylalanines 41 suspended in aqueous solution with proteinase K is shown. Here, image A) has been taken at time 0 minutes, image B) at 20 minutes, C) at 40 minutes and image D) after 60 minutes after adding proteinase K to the solution. As can be readily seen, the SHG signal generated by the self-assembled structures of triphenylalanines 41 is decreasing over the course of time, indicating the digestion 201 or decomposition of the structure.

FIG. 7 shows that the nanoparticles 1 comprising a polymer shell 2 are not aggregating over time (compare to FIG. 4). The nanoparticles 1 are suspended in an aqueous solution 12 and have been prepared according to the method according to the invention. The such obtained nanoparticles 1 comprise a PLLA coating that encloses a structured plurality 40 of triphenylalanines 41, that give rise to the detectable SHG signal. Image A) has been taken at time 0 minutes, image B) after 20 minutes, image C) after 40 minutes and image D) after 60 minutes. As can be seen, the SHG signal is not clustering over the course of time, which would indicate aggregation. Thus, the polymer shell 2 indeed prevents the aggregation of the self-assembled peptides 4.

FIG. 8 shows the molecular structures of cyclo(-D-Trp-Tyr) 50 on the top panel and cyclo(-Phe-Phe) 51 on the bottom panel.

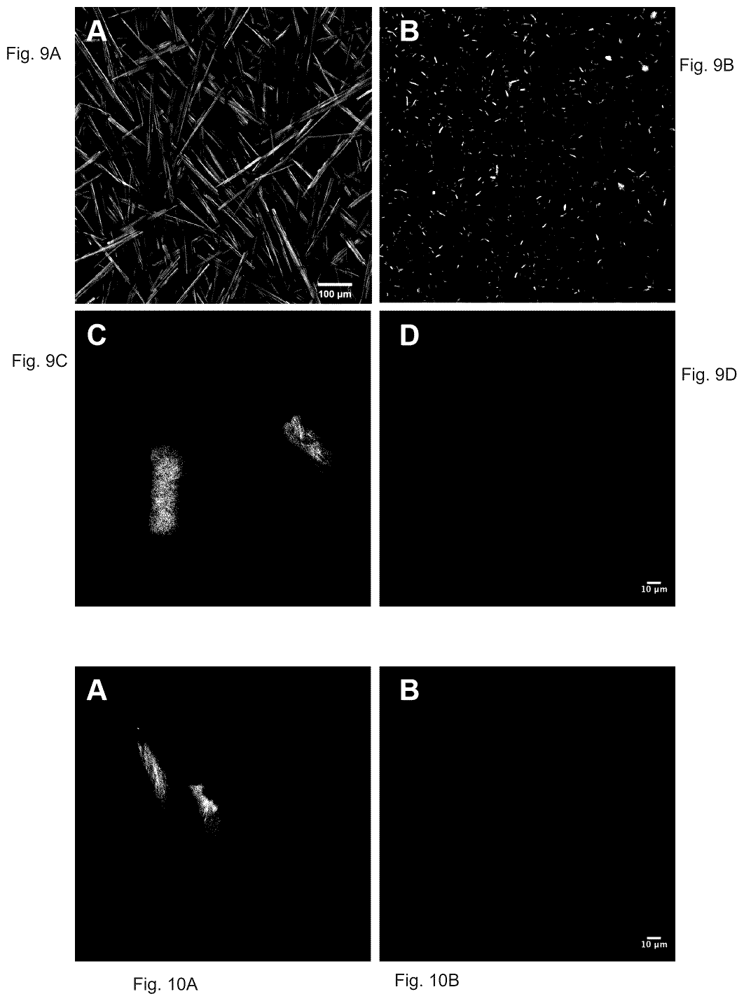

FIG. 9 shows various SHG images. The white regions in the images indicate the presence of an SHG signal, wherein the black regions indicate the absence of an SHG signal. Panel A: State of the art large scale peptide nanotubes using diphenylalanine (FF) peptide. Such large scale assemblies are formed when no shell layer is provided to inhibit the growth of the peptides. Panel B: In contrast to the state of the art, encapsulated triphenylalanine (FFF) peptide based on the emulsion-solvent evaporation method according to the invention form stable nanoparticles that generate a SHG signal upon illumination. Panel C: LIVAGK peptide assemblies described by [4] show large-scale peptide assemblies. Panel D: Nonetheless, when LIVAGK peptides are encapsulated, the SHG signal vanishes.

FIG. 10 shows two SHG images of LIVAGK assemblies in two different concentrations. Panel A: LIVAGK peptides' SHG signal at a concentration of 100 mg/ml. Panel B: LIVAGK peptides' SHG signal at a concentration of 10 mg/ml. In panel B no SHG signal can be observed, as the aggregated peptides have disassembled.

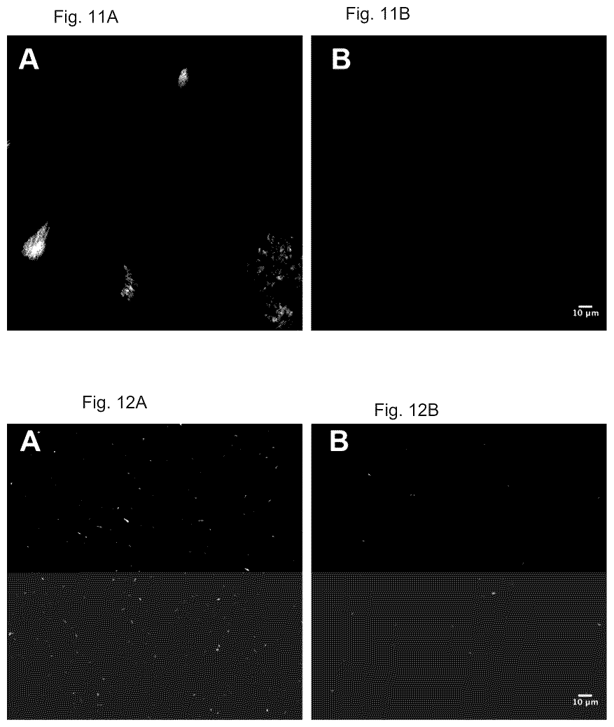

FIG. 11 shows two SHG images of LIVAGK assemblies in a solution with a pH-value of 7 (panel A) or pH 4 (panel B) at a concentration of 100 mg/ml. While in panel A the peptides are still assembled and give rise to an SHG signal, the peptides disassemble at pH 4, such that the SHG signal cannot be observed anymore. The disassembly occurs within minutes (˜5 minutes) after incubation in pH 4.

FIG. 12 shows two SHG images of nanoparticles according to the invention. Panel A: shows the nanoparticles' SHG signal at a concentration of 1.5 mg/ml. Panel B shows the SHG signal of the nanoparticles at a concentration of 0.15 mg/ml. The nanoparticles do not suffer from dissociation or disassembly but remain stable. The SHG signal persists.

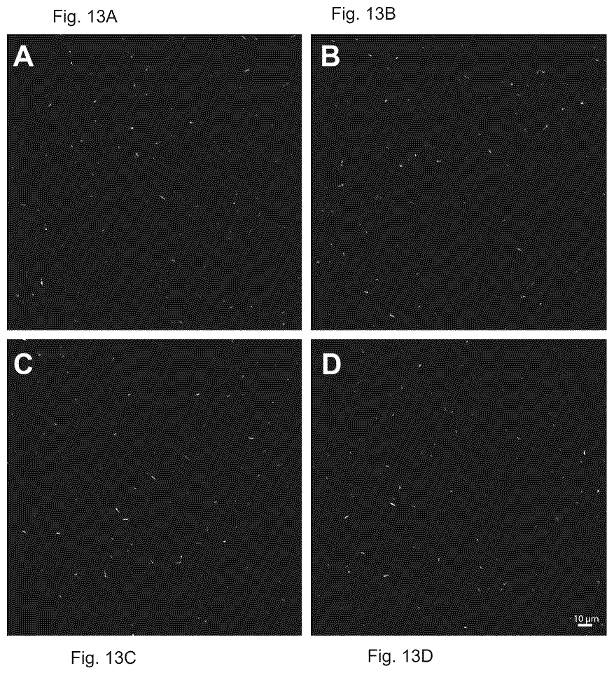

FIG. 13 shows SHG images of the nanoparticles according to the invention in various solutions with different pH-values. Panel A: SHG signal after incubation of the nanoparticles for 72 hours in a buffer at pH 7. Same as in panel A, but at pH 6. Same as in panel A, but at pH 5. Panel D: same as in panel A, but at pH 4.

The method may alternatively be described and claimed as follows:

A method for producing second harmonic generation (SHG)-active biodegradable nanoparticles, comprising:
dissolving a self-assembling SHG-active and/or THG active peptide and a biodegradable polymer in an organic solvent to form an organic dispersed phase,
emulsifying said organic phase into an aqueous phase containing a surfactant to generate an oil-in-water emulsion,
particularly sonicating the emulsion to reduce droplet size and/or to stabilize the emulsion;
removing the organic solvent, particularly by evaporation, particularly under stirring, wherein the peptide undergoes self-assembly and crystallizes in situ within the droplets during solvent evaporation, resulting in nanoparticles comprising a crystalline peptide core encapsulated within a biodegradable polymer shell and stabilized by a surfactant layer.

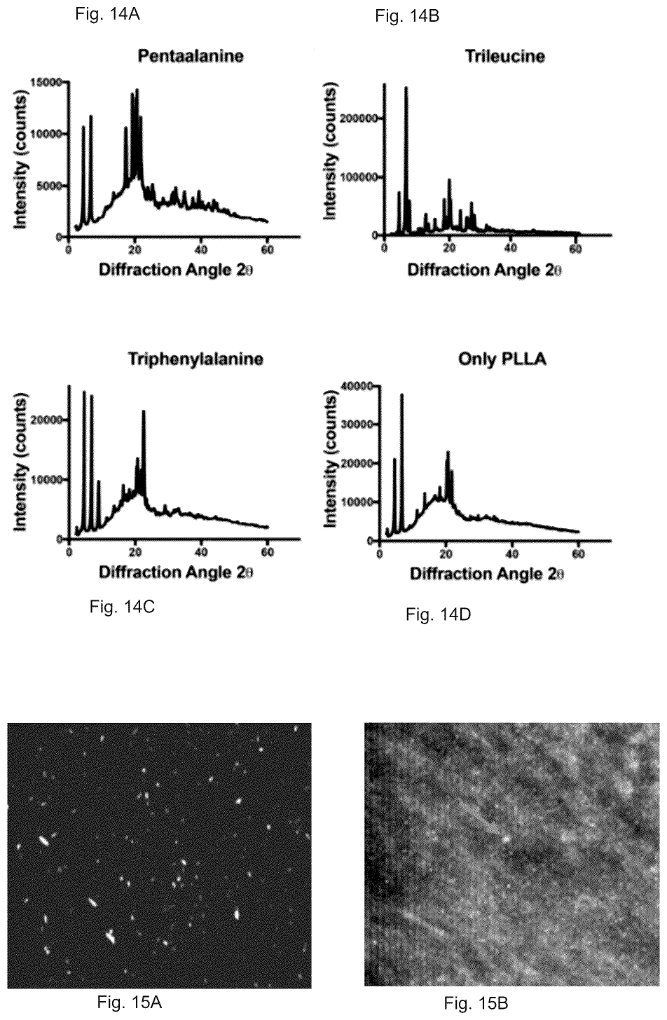

FIG. 14 shows the results of the method according to the invention using different peptides for the manufacturing the nanoparticles.

The recorded XRD patterns of the nanoparticles with different peptide cores show distinct diffraction patterns and high degree of internal order associated with their individual crystalline phases. a) Pentaalanine, b) Trileucine, c) Triphenylalanine, d) nanoparticles without a peptide core display significant background with overlapping peaks, interfering with a definitive crystallographic analysis of peptide core.

FIG. 15 shows the capability of the nanoparticles to generate a third harmonic signal.

All images captured with 3-photon excitation at 1300 nm. The third harmonic signal was collected at 433 nm using 20 nm band emission filter. The nanoparticles were immobilised in 2% agarose gel. Panel A) 130×150 um field of view image showing dispersed nanoparticles each giving rise to e THG signal upon illumination. Panel B) the nanoparticles imaged through 100 μm thick slice of brain tissue. Probe visible in center of the image (grey arrow).

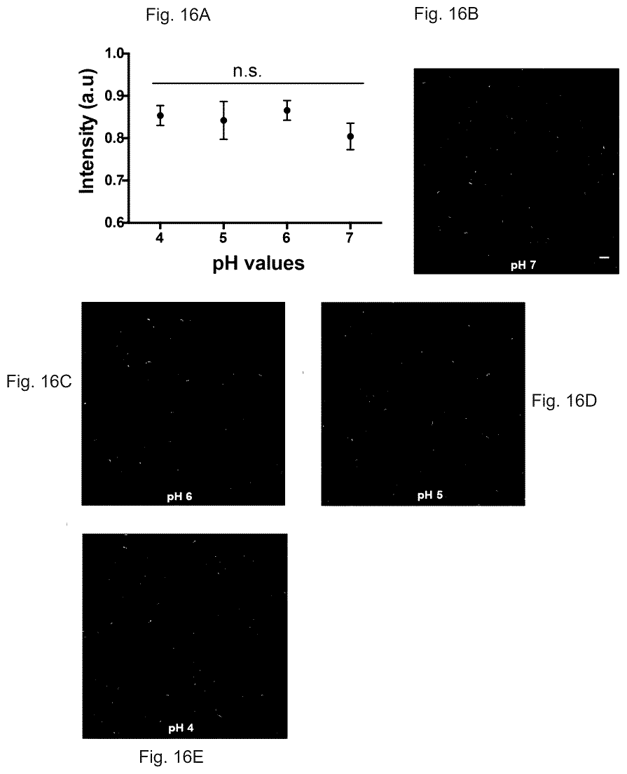

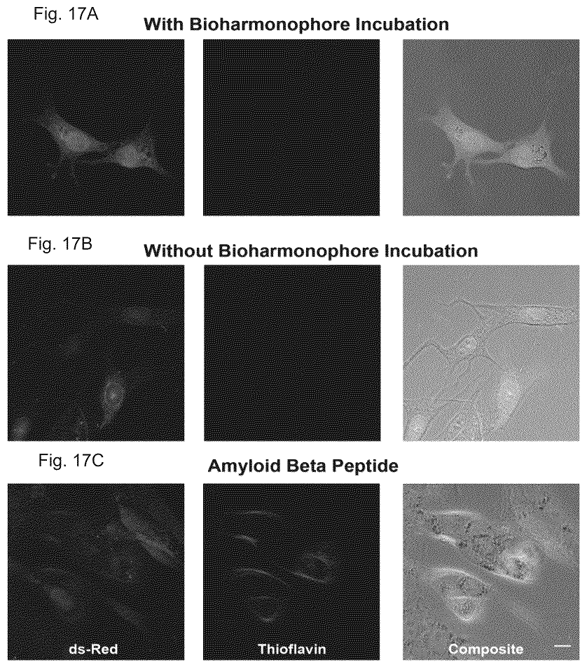

FIG. 16 shows the emission of nanoparticles under different pH conditions. Lower pH does not affect the signal intensity of the nanoparticles. In FIG. 16a, the effect of different pH values on the SHG signal intensity are shown as a diagram. The nanoparticles were incubated at different pH values for 72 hours (n=5). Representative images showing SHG signal intensity after the nanoparticles were incubated in buffers at FIG. 16b, pH 7, FIG. 16c, pH 6, FIG. 16d, pH 5, and FIG. 16e, pH 4. Scalebar, 10 μm. Mean±s.d. n.s., not significant (Ordinary one-way ANOVA with Tukey's multiple comparisons).

In FIG. 17 is demonstrated that the nanoparticles do not induce misfolding of proteins in living cells. Panels a) to c) if FIG. 17 show a Thioflavin T staining of DsRed expressing MDA-MB-435 cancer cells. The staining reveals that the incubation with the nanoparticles does not induce protein misfolding. FIG. 17a, shows a DsRed-expressing cancer cells that were incubated with the nanoparticles, cultured for one day, and stained with Thioflavin T to detect any nanoparticle-induced aggregation toxicity. (left) DsRed, (center) Thioflavin T signal, (right) composite image. FIG. 17b, shows DsRed-expressing cancer cells cultured without the nanoparticles, followed by Thioflavin T staining. (left) DsRed, (center) Thioflavin T signal, (right) composite image. The absence of Thioflavin T signal indicates that cells incubated with the nanoparticles do not display any protein aggregation-induced toxicity, similar to control cells in FIG. 17a. FIG. 17c, as a positive control, cells were incubated with amyloid beta protein for 24 hours and stained following the same protocol. Scalebar, 10 μm.

FIG. 18 shows that the nanoparticles do not cause toxicity to cells and zebrafish embryos. FIG. 18a, from left to right, the nanoparticles were coated with p32 peptide, PEG, and PLLA and incubated with cells for 48 and 72 hours. Under these conditions, they do not affect cell viability (n=3). FIG. 18b, the nanoparticles were injected into the zebrafish blood stream at 2 dpf and their survival rate was recorded at 5 dpf. Under these conditions, they do not harm the zebrafish embryos (N=75, pooled from 3 independent experiments). Mean±s.d., n.s., not significant (Ordinary one-way ANOVA with Tukey's multiple comparisons).

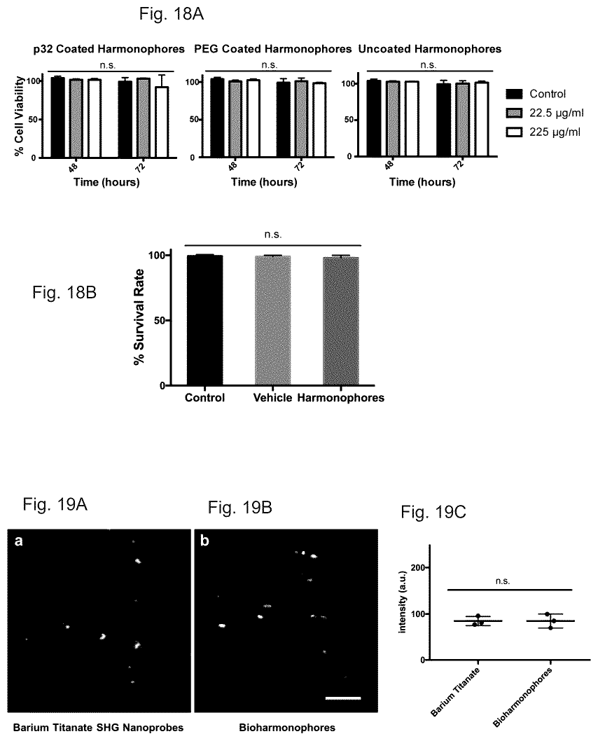

FIG. 19 shows a comparison between inorganic SHG nanoparticles and the biodegradable nanoparticles. Both nanoparticles show comparable signal intensities under the same illumination conditions. Similar size ranges of barium titanate SHG nanoprobes and the nanoparticles according to the invention were illuminated with the same laser intensity and their SHG signal was monitored using the same detection settings. FIG. 19 a,b, SHG images of both nanoparticles display comparable particle brightness for inorganic SHG nanoprobes (left) and the biodegradable nanoparticles (right) regarding signal-to-noise ratio and single particle sensitivity. Scalebar, 10 μm. FIG. 19c, provides a quantification of single particle intensities demonstrating that there is no statistically significant difference between the signal intensities of SHG nanoprobes and nanoparticles according to the invention, respectively.

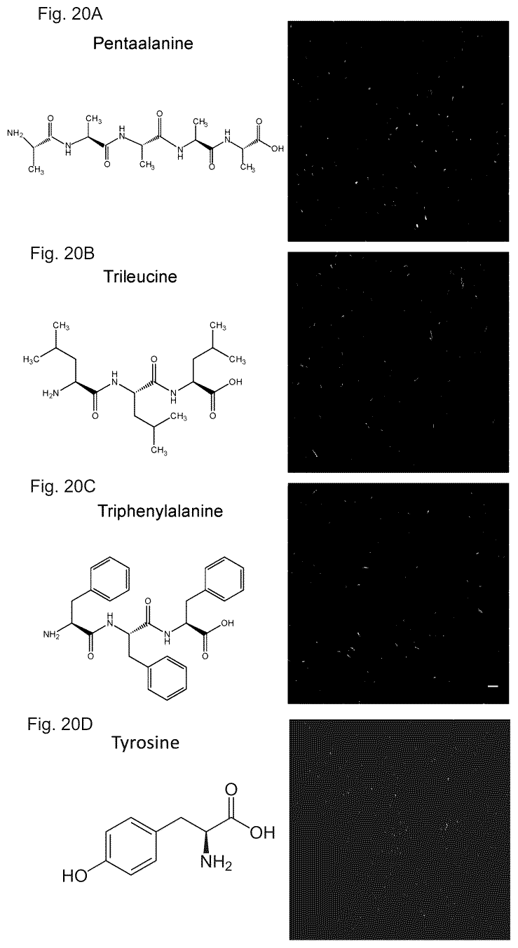

In FIG. 20 shows the SHG signal of the nanoparticles comprising different self-assembling peptides at the core. The chemical structure of FIG. 20a, Pentaalanine, FIG. 20b, Trileucine, FIG. 20c, Triphenylalanine, and FIG. 20d, Tyrosine peptides (left) and corresponding images capturing their SHG signal after encapsulation with the polymer shell (right). Scalebar, 10 μm.

FIG. 21 shows an SHG emission pattern of nanoparticles comprising a peptide core with a structure plurlati yof Triphenylalanine. The SHG emission pattern indicates monolithic core-shell nanoparticles. FIG. 21a: Arrow 2100 indicates an excitation beam direction. Arrow 2101 shows a SHG collection direction, which rotates between −90° and +90°. The detected polarization is in the beam's plane (P, arrow 2102). The pattern 2103 shows PPP polarization configuration (excitation and detection polarizations in the plane of the beams), and the pattern 2104 shows PSS (excitation with a perpendicular polarization). FIG. 21b: SHG intensity vs incident polarization angle for a nanoparticle. Pattern 2105 shows detection along the X axis while pattern 2106 shows detection along the Y axis. The solid lines forming the patterns correspond to a fitted curve, assuming C₂ symmetry. The massive circles represent the measured values.

### Examples

### Example 1: Preparation of a Suspension of Nanoparticles

In the following a brief, non-limiting example of the preparation of a suspension of nanoparticles according to the invention is given:

Dissolve 5 mg Phe-Phe-Phe and 30 mg Poly-Lactic Acid in 3 ml Chloroform.

Mix the peptide-polymer solution with 10 ml SDS aqueous solution (1 mg/ml) as surfactant.

After stirring the mixture for 1 hour, sonicate the sample using a Probe Sonicator for 2 minutes at 70% power.

Stir the suspension overnight at 37° C. to evaporate chloroform and so that the aqueous suspension of nanoparticles according to the invention is obtained.

While the actual steps remain particularly the same, the concentrations of peptide, polymer and surfactant might be different when using a different peptide.

With regard to the method for manufacturing the nanoparticles according to the invention the following exemplary manfacturing protocol is disclosed.

The biodegradable nanoparticle is produced by co-encapsulating peptide and polymer precursors in an oil-in-water miniemulsion, whereby the self-assembly and crystallization of the SHG-active and/or THG active peptide occurs in situ within the encapsulated droplets during solvent evaporation. The process yields stable, monodisperse, second and third harmonic generating nanoparticles suitable for in vivo use.

### Materials

Self-assembling peptide: e.g., Triphenylalanine (FFF)

Biodegradable polymer: Poly(L-lactic acid) (PLLA)

Organic solvent: Chloroform

Surfactant: Sodium dodecyl sulfate (SDS)

Deionized water and Tween 80 for post-processing.

Equipment: Sonicator (Branson Sonifier), stirrer, centrifuge, ice bath and dynamic light scattering (DLS) setup.

Organic Phase Preparation-Dissolve:
15 mg of Triphenylalanine (FFF) peptide,
30 mg of Poly(L-lactic acid) (PLLA),
in 3 mL of chloroform to form the organic dispersed phase.

Aqueous Phase Preparation-Prepare an aqueous solution containing 0.3% (w/v) sodium dodecyl sulfate (SDS).

Emulsification—Add the organic phase to the aqueous SDS solution and stir at 1000 rpm for 1 hour to form a pre-emulsion.

Miniemulsion Formation—Subject the emulsion to probe sonication (e.g., 70% amplitude, pulsed 30 s ON/10 s OFF, for 2 minutes) while cooling on an ice bath to prevent thermal denaturation.

### In Situ Peptide Crystallization and Solvent Evaporation

Stir the emulsion overnight at 40° C. and 500 rpm. During this step:
The organic solvent evaporates,
The polymer precipitates to form a shell,
The peptide self-assembles and crystallizes in situ within the polymeric droplets to form the functional core.

### Results

Yields stable nanoparticles of <200 nm comprising:
a crystalline peptide core, a biodegradable PLLA shell, a surfactant droplet layer on the surface.

Optimization Parameters

| Component | Optimal Value | Rationale |
|---|---|---|
| Peptide | 15 mg (33 wt %) | Highest SHG with good stability; 40 wt % |
| (FFF) | | aggregates; 25 wt % gives low signal. |
| PLLA | 30 mg (66 wt %) | Ensures sufficient encapsulation and optimal |
| | | morphology; 86 wt % yields elongated particles. |
| SDS | 0.3% (40 wt % of | Achieves stable dispersion and optimal SHG; low |
| | dispersed phase) | (18 wt %) leads to aggregation; high (57 wt %) |
| | | reduces SHG due to small size. |

It is noted that according to the invention in situ formation of crystalline SHG-active and/or THG active peptide inside nanocapsules, i.e. the droplets during solvent evaporation is not pre-formed or separately crystallised prior to encapsulation.

The self-assembly is confined within microenvironments, provided by the droplets in the emulsion which enhances size uniformity, signal reproducibility, and biocompatibility.

### REFERENCES

[1] Boyd, R. W. Nonlinear optics (Academic Press, 2013)
[2] Kholkin, A., et al. “Strong piezoelectricity in bioinspired peptide nanotubes”, ACS Nano 4, 610-614 (2010)
[3] Handelmann, A. et al. “Nonlinear optical bioinspired peptides nanostructures” Advanced Optical Materials 1, 875-884 (2013)
[4] WO 2016/007091 A1

We claim:

1. A method for preparing an aqueous suspension of biodegradable, water-suspendable nanoparticles (1) with a peptide core and a polymer shell, wherein the nanoparticles (1) each provide a second-harmonic signal and/or a third-harmonic signal upon illumination, the method comprising the steps:
providing an organic phase (10) comprising an organic solvent with peptides (4) and a biodegradable polymer (3) and/or monomers that form the polymer (3) upon polymerization,
providing an aqueous phase (12) comprising an aqueous solution with a surfactant (11),
preparing (100) a miniemulsion (14) of the organic phase (10) and the aqueous phase (12), wherein the miniemulsion (14) comprises droplets (13) of the organic phase (10) emulsified in the aqueous phase (12), wherein a majority of the peptides (4) are comprised in the droplets of the organic phase,
self-assembling the peptides into at least one structured plurality (40) of peptides (4) in the droplets, wherein said structured plurality (40) of peptides generates a second-harmonic signal and a third-harmonic signal upon illumination and forms the peptide core of the nanoparticle,
creating a polymer shell for each nanoparticle, said polymer shell encapsulating and maintaining the structured plurality of peptides, wherein creating the polymer shell comprises removing (101) the organic solvent from the miniemulsion (14) to obtain the aqueous suspension of biodegradable, water-suspendable nanoparticles.

2. The method according to claim 1, wherein the organic phase is a water-immiscible solvent.

3. The method according to claim 1, wherein the miniemulsion (14) is prepared by applying shear-forces to the mixed solution of the organic phase (10) and aqueous phase (12).

4. The method according to claim 3, wherein the shear-forces are applied by sonication of the mixed solution.

5. The method according to claim 1, wherein the organic solvent is removed (101) from the miniemulsion (14) by evaporating the organic solvent.

6. The method according to claim 1, wherein the organic solvent is chloroform, the peptide (4) comprises triphenylalanine (41) and the polymers (3) comprise Poly-Lactic Acid and the aqueous phase (12) comprises sodium dodecyl sulfate as surfactant (11).

7. The method according to claim 1, wherein the organic phase comprises monomers that are polymerized in a polymerization step to form polymers.

8. The method according to claim 1, wherein a majority of the droplets of the miniemulsion have a diameter in the range between 50 nm to 250 nm.

9. The method according to claim 1, wherein the structured plurality of peptides comprises a non-centrosymmetric crystallized amino acid-based or peptide-based molecules selected from the group of consisting of:
mono-amino acids, i.e. monopeptides
dipeptides,
tripeptides, and
oligopeptides comprising more than three amino acids.

10. The method according to claim 1, wherein the polymers are selected from the group consisting of:
polylactic acid, (PLA),
polyglutamic acid (PGA), and
polycaprolactone (PCL).

11. The method according to claim 1, wherein the steps of self-assembling of the peptides and creating a polymer shell are executed by way of a co-crystallization of the peptide core into the structured plurality of the peptides within a matrix made of the polymers.

12. The method according to claim 1, wherein the nanoparticles have a median diameter of less than 200 nm.

13. The method according to claim 1, wherein the peptides crystallize forming the structured plurality of peptides in the peptide core during the self-assembling step and during the removal of the organic solvent from the miniemulsion.

14. The method according to claim 13, wherein the peptides crystallize forming a lattice of a non-centrosymmetric space group selected from the group of chiral or polar space groups including C₂, D₂, P6₁, monoclinic space groups.

15. The method according to claim 1, wherein a second harmonic signal intensity is equivalent to or greater than that of inorganic second-harmonic generating materials under the same imaging conditions, including but not limited to barium titanate (BaTiO₃).

16. The method according to claim 1, wherein the nanoparticles are stable in the aqueous suspension for at least 48 hours.

17. The method according to claim 1, wherein the nanoparticles maintain the second- and the third harmonic signal across a pH range ranging from pH 0 to pH 14, particularly ranging from pH 4 to pH 10.

18. The method according to claim 1, wherein the nanoparticles are non-toxic to cells and organisms under in vitro and in vivo conditions.

19. The method according to claim 1, wherein nanoparticles do not form 3-sheet aggregates or fibrillar structures.

20. A method for second-harmonic generation imaging of a sample comprising a nanoparticle (1) manufactured according to claim 1, comprising the steps of:
providing a sample with the nanoparticle (1),
illuminating the sample with light comprising a first wavelength for second or third harmonic generation
detecting the light stemming from the second or the third harmonic generation.
